(19) United States
(12) Patent Application Publication
(10) Pub. No.: US 2023/0414912 A1
(43) Pub. Date: Dec. 28, 2023

(54) Microneedle Array Coated with Composition Containing Particles

(71) Applicant: MEDRx Co., Ltd., Kagawa (JP)

(72) Inventors: Hidetoshi Hamamoto, Higashikagawa-shi, Kagawa (JP); Masaki Ishibashi, Higashikagawa-shi, Kagawa (JP); Takashi Nakae, Higashikagawa-shi, Kagawa (JP); Haruka Kawahara, Higashikagawa-shi, Kagawa (JP); Jun Nakamura, Higashikagawa-shi, Kagawa (JP)

(73) Assignee: MEDRx Co., Ltd., Kagawa (JP)

(21) Appl. No.: 18/036,637

(22) PCT Filed: Nov. 11, 2021
(86) PCT No.: PCT/JP2021/041530

§ 371 Date: May 11, 2023

(30) Foreign Application Priority Data
Nov. 12, 2020 (JP) 2020-188700

(51) Int. Cl. A61M 37/00

(52) U.S. Cl. CPC A61M 37/0015; A61M 2037/0053

(57) ABSTRACT

The present invention provides a microneedle array having microneedles coated with a composition comprising a poorly water-soluble or water-insoluble particle with a mean particle size of 1 μm or less, 2% to 20% by weight of a binding agent and 60% by weight or more of water in which the poorly water-soluble or water-insoluble particle is loaded on the microneedles in equal amounts that shows good puncturability.

### TECHNICAL FIELD

The present invention relates to a microneedle array coated with a composition comprising a particle, specifically a microneedle array having microneedles coated with a composition comprising a particle.

### BACKGROUND ART

Recently, a microneedle array has been frequently tried to be used as one of transdermal administration methods of a drug. As one of the methods of fixing a drug on a microneedle array, the attempt to coat a solution comprising a drug on microneedles has been made. The solution coated on the microneedles in the microneedle array is required to have not only the flowability to be easily coated on fine microneedles but also adequate viscosity and drying properties so that the solution is loaded without being removed from the microneedles and the microneedles have tip shapes with good punctureability after drying.

As a method of giving such properties to a coating solution for microneedles, there is the method of adding an additive into the solution. However, the type of additives is limited because it is required to ensure the compatibility with drugs and the same safety as aqueous injection formulations. For example, it has been reported in Patent Document 1 that it is preferable to use a substance that is compatible (having the property of being homogeneously mixed) with active ingredients such as follicle stimulating hormone as a coating carrier for microneedles made of polylactic acid resin.

However, a microneedle array loaded with a poorly water-soluble or water-insoluble particle having no property of being homogeneously mixed have not been put to practical use.

### PRIOR ART DOCUMENTS

### Patent Documents

Patent Document 1: WO 2010-074239

### SUMMARY OF INVENTION

### Problem to be Solved by the Invention

An object of the present invention is to provide a microneedle array having microneedles loaded with a poorly water-soluble or water-insoluble particle in equal amounts that shows good puncturability.

### Means for Solving the Problem

The present inventors observed that when a solution comprising a poorly water-soluble drug was coated on microneedles, there was a large variation in the amount of drug loaded besides the roughening of each microneedle surface. When the microneedle surface is roughened, there are the possibilities of reducing the puncturability of microneedles and removing the coated drug from the microneedles when punctured into the skin. In addition, the methods that cause variation in the amount of drug loaded are not suitable for the preparation of pharmaceutical formulations.

The present inventors have extensively studied to reach the above object, and then have found that when a particle with a mean particle size below a predetermined value is used and a solution comprising it is prepared with predetermined compositions, microneedles having sharp tip parts and smooth surfaces that show good reproducibility in the amount of particle loaded even when a particle such as poorly water-soluble drug is used. Based on the findings, the present invention has been completed.

That is, the present invention provides the following embodiments.

(1) A microneedle array having microneedles coated with a composition comprising:
a poorly water-soluble or water-insoluble particle with a mean particle size of 1 μm or less,
2% to 20% by weight of a binding agent, and
60% by weight or more of water.
   (2) The microneedle array according to the above item (1), wherein the binding agent is one or more selected from the group consisting of a saccharide or a derivative thereof, a protein additive, a polyvinyl alcohol compound, a polyacrylic acid compound, a polyglycolic acid compound, a polyamide compound, a polyester compound, and polyvinyl pyrrolidone.
   (3) The microneedle array according to the above item (2), the saccharide or a derivative thereof comprises one or more saccharides selected from the group consisting of hydroxypropyl cellulose, sodium carboxymethyl cellulose, trehalose and sucrose.
   (4) The microneedle array according to any one of the items (1) to (3), wherein the poorly water-soluble or water-insoluble particle has a mean particle size of 700 nm or less.
   (5) The microneedle array according to any one of the items (1) to (3), wherein the poorly water-soluble or water-insoluble particle has a mean particle size of 500 nm or less.
   (6) The microneedle array according to any one of the items (1) to (5), wherein the poorly water-soluble or water-insoluble particle has a mean particle size of 50 nm or more.
   (7) The microneedle array according to any one of the items (1) to (6), wherein the poorly water-soluble or water-insoluble particle is a drug selected from indomethacin, diclofenac, flurbiprofen, etodolac, fentanyl, lidocaine, apomorphine, donepezil, buprenorphine, naproxen, meloxicam, estradiol, progesterone, methaxalone, cyclosporine, celecoxib, cilostazol, ciprofloxacin, or a salt thereof.
   (8) The microneedle array according to any one of the items (1) to (7), wherein the poorly water-soluble or water-insoluble particle is pre-milled particle.
   (9) The microneedle array according to any one of the items (1) to (8), wherein the amount of the poorly water-soluble or water-insoluble particle is 60% or more relative to the total amount of the composition excluding water.
   (10) A method of manufacturing a microneedle array, which comprises:
milling a poorly water-soluble or water-insoluble compound until the mean particle size of the compound is 1 μm or less,
preparing a composition comprising the milled poorly water-soluble or water-insoluble compound, 60% by weight or more of water and 2% to 20% by weight of a binding agent, and
coating the composition on microneedles.
   (11) The method according to the above item (10), wherein the milling of the poorly water-soluble or water-insoluble compound is wet milling with at least one mill selected from the group of a jet mill, a bead mill and a planetary mill.
   (12) A method of manufacturing a microneedle array, which comprises:
pouring a composition comprising a poorly water-soluble or water-insoluble particle with a mean particle size of 1 μm or less, water, and a binding agent into a mold with a concave portion forming the form of microneedles,
evaporating water to form a microneedle array, and
removing the formed microneedle array from the mold.
   (13) The method according to the above item (12), wherein the composition comprises water in an amount of 60% by weight or more and the binding agent in an amount of 2% to 20% by weight.
   (14) The method according to the above item (12) or (13), which comprises milling a poorly water-soluble or water-insoluble compound to obtain the poorly water-soluble or water-insoluble particle with a mean particle size of 1 μm or less before pouring the composition into the mold.
   (15) The method according to the above item (14), wherein the milling of the poorly water-soluble or water-insoluble compound is wet milling with at least one mill selected from the group of a jet mill, a bead mill and a planetary mill.
   (16) A microneedle array manufactured by the method according to any one of the above items (12) to (15).
   (17) A microneedle array having microneedles incorporating a poorly water-soluble or water-insoluble particle with a mean particle size of 1 μm or less and a binding agent.

### Effects of the Invention

The microneedle array of the present invention has less variation in the amount of particle loaded and has microneedles with smooth surfaces and sharp tip parts even when coated the microneedles with a composition comprising a poorly water-soluble or water-insoluble particle. Thus, the microneedle array is suitable as pharmaceutical formulations and shows good puncturability.

### BRIEF DESCRIPTION OF THE DRAWINGS

FIG. 1 is a graph showing the particle size distribution after indomethacin is milled by a planetary ball mill.

FIG. 2 is a magnified photograph of tip parts of microneedles coated with a composition comprising milled or unmilled indomethacin (×600 folds). FIG. 2A shows the tip part of a microneedle coated with the composition comprising milled indomethacin, and FIG. 2B shows the tip part of a microneedle coated with the composition comprising unmilled indomethacin.

FIG. 3 is a graph showing the variation in the amount of milled or unmilled indomethacin loaded on a microneedle array. • shows the amount of the composition comprising unmilled indomethacin loaded, ∘ shows the amount of the composition comprising milled indomethacin loaded, and - shows the average value of each indomethacin loaded (left side: unmilled, right side: milled).

### DESCRIPTION OF EMBODIMENTS

The first embodiment of the present invention relates to a microneedle array having microneedles coated with a composition comprising a poorly water-soluble or water-insoluble particle with a mean particle size of 1 μm or less, 60% by weight or more of water, 5% to 10% by weight of a binding agent.

In one embodiment, the “microneedle array” is a microneedle array having about 50 to about 1000 fine needles (microneedles) with a height of about 300 μm to about 1000 μm per cm² on the base. The microneedle array used herein may be made of a material comprising a resin, a ceramics and a metal. Furthermore, the material of the microneedle array may be a material comprising a thermoplastic resin. Preferably, the material is a material comprising a biodegradable thermoplastic resin. It is expected that such material can be easily produced on a large scale, and can adequately assure the safety in use.

The form of each microneedle is not particularly limited. For example, the form may be in the form of polyglonal pyramid (e.g., triangular pyramid, quadrangular pyramid, hexagonal pyramid) having a cross-sectional area that decreases toward the tip or conical pyramid. The cross-sectional area herein may be configured to be small continuously and progressively toward the tip or to be discontinuously small in one or more positions. The form of each microneedle may have a forward end portion in the form of triangular pyramid, quadrangular pyramid, hexagonal pyramidor conical pyramid on a frustum such as triangular truncated pyramid, quadrangular truncated pyramid, hexagonal truncated pyramid and conical truncated pyramid, as described in WO 2012/057345. Also, the form of each microneedle may be a two-stage needle having a base section such as triangular truncated pyramid, quadrangular truncated pyramid, hexagonal truncated pyramid and conical truncated pyramid and a shaft section formed on the base section in which the area of the top surface of the base section is larger than the area of the bottom surface of the shaft section, as described in WO 2013/162053.

In one embodiment, the “poorly water-soluble or water-insoluble particle” comprised in a composition coated on microneedles is a poorly water-soluble or water-insoluble drug. The term “poorly water-soluble” or “water-insoluble” is interpreted in the broadest sense. For example, the term is understood to be “sparingly soluble” to “practically insoluble” defined in the Japanese Pharmacopoeia 17th edition, that is, mean that the volume of solvent required for dissolving 1 g of solute is 30 mL or more.

The poorly water-soluble or water-insoluble drug may be selected from, for example, indomethacin, diclofenac, flurbiprofen, etodolac, fentanyl, lidocaine, apomorphine, donepezil, buprenorphine, naproxen, meloxicam, estradiol, progesterone, methaxalone, cyclosporine, celecoxib, cilostazol, ciprofloxacin, or a salt thereof.

The “poorly water-soluble or water-insoluble particle” may be a compound other than drug. For example, a microparticle such as a lipid, a resin and a metal may be used at appropriate.

In one embodiment, the “poorly water-soluble or water-insoluble particle” has a mean particle size of about 1 μm or less. The range of the mean particle size ensures that the variation in the amount of particle loaded on a microneedle array is sufficiently small. The variation in the amount of particle loaded among microneedle arrays can be evaluated by, for example, coefficient of variation (CV). CV is not particularly limited, but is preferably 10% or less and more preferably 5% or less. Also, a microneedle array having microneedles with smooth surfaces and sharp tips after coating a composition thereon can be manufactured by the use of a “poorly water-soluble or water-insoluble particle” with a mean particle size of 1 μm or less. The mean particle size thereof is preferably about 700 nm or less and more preferably about 500 nm or less. Also, the mean particle size thereof may be about 50 nm or more or about 100 nm or less. The mean particle size thereof can be measured according to a well-known method or a method equivalent thereto. When the mean particle size thereof measured by any one of the methods is 1 μm or less, they are included in the scope of the present invention.

When the mean particle size of a poorly water-soluble or water-insoluble compound such as a poorly water-soluble or water-insoluble drug bulk powder exceeds 1 μm, the compound may be pre-milled to adjust the mean particle size thereof to 1 μm or less. The milling method may be appropriately selected depending on the type of poorly water-soluble or water-insoluble compounds. The method may be dry milling or wet milling. As the milling mechanism, compression, impact, shear, friction, or two or more combination thereof may be used. A mill such as a jaw crusher, a gyratory crusher, a crushing roll, a hammer mill, a roller mill, a jet mill, a ball mill, a vibration ball mill, a planetary mill and a bead mill may be used depending on the respective mechanism. In particular, the wet milling with a jet mill, a planetary mill or bead mill can mill the target to nanosize.

In one embodiment, the amount of the “poorly water-soluble or water-insoluble particle” in the composition is not particularly limited. For example, the amount thereof may be, for example, 1 to 30% by weight, and preferably 5 to 25% by weight.

In one embodiment, the term “binding agent” is used in the broadest sense. For example, it may be one or more selected from a saccharide or a derivative thereof, a protein additive, a polyvinyl alcohol compound, a polyacrylic acid compound, a polyglycolic acid compound, a polyamide compound, a polyester compound, and polyvinyl pyrrolidone.

As the “saccharide or a derivative thereof”, one or more saccharides or derivatives thereof selected from the group consisting of, for example, alginic acid, agar, starch, hydroxypropyl cellulose, sodium carboxymethyl cellulose, hyaluronic acid, trehalose, lactose, sucrose, fructose, galactose, mannose, maltose, gulucose, mannitol, pullulan, sorbitol, and dextran may be used. Preferably, one or more saccharides selected from the group consisting of hydroxypropyl cellulose, sodium carboxymethyl cellulose, trehalose, and sucrose are used. The addition of the saccharide or a derivative thereof attracts surrounding water into a microneedle array due to the difference in osmotic pressure after the microneedle array is punctured into the skin, and thus can enhance the release of drug from the microneedle array.

As the “protein additive”, a protein such as albumin, casein, gelatin and collagen may be used.

In one embodiment, the amount of the “binding agent” in the composition is not particularly limited. For example, the amount thereof is about 2% to about 20% by weight and preferably about 3% to about 10% by weight. When the amount of the binding agent in the composition is below about 2% by weight, there is a tendency to make it difficult to load the composition on microneedles, and the composition tends to be cracked or be removed from the microneedles easily after the composition is coated on the microneedles and dried. When the amount of the binding agent in the composition exceeds about 20% by weight, there is a tendency to make it impossible to coat the composition on the microneedles due to too high viscosity and to not sufficiently increase the ratio of particles in the composition.

In one embodiment, the composition comprises about 60% by weight or more of water. The amount of water in the composition is preferably about 70% by weight or more. The composition may comprise a solvent other than water as long as the effects of the present invention are not blocked. In view of the drying property, it is preferable to use only water as the solvent.

Examples of the solvent other than water include a water-soluble solvent such as an alcohol, for example, ethyl alcohol, isopropyl alcohol and an organic solvent (e.g., an ether such as tetrahydrofuran, an ester such ethyl acetate) in an amount that can be mixed.

When the composition comprises a solvent other than water, the ratio of water in the solvent is preferably 90% by weight or more and more preferably 95% by weight or more. In one embodiment, the composition may further comprise an additive such as a pH adjuster, an antioxidant, and a preservative as long as the effects of the present invention are not blocked. As the above suitable additive, a commercially available reagent may be used for any purpose.

Examples of the pH adjuster include a buffering agent consisting of an organic acid such as citric acid, tartaric acid, lactic acid, fumaric acid, and malic acid and an alkali metal salt thereof, and a buffering agent consisting of an inorganic acid such as phosphoric acid and an alkali metal salt thereof.

Examples of the antioxidant include ascorbic acid, dibutylhydroxytoluene (BHT), sodium hydrogen sulfite, sodium sulfite, erythorbic acid, tocopherol acetate, dibutylhydroxytoluene, tocopherol, sodium pyrosulfite, butylhydroxyanisol, and propyl gallate.

Examples of the preservative include benzoic acid, sodium benzoate, sorbic acid, sodium sorbate, sodium dehydroacetate, paraoxybenzoic acid, sodium paraoxybenzoate, ethyl paraoxybenzoate, propyl paraoxybenzoate (propylparaben), butyl paraoxybenzoate, isopropyl paraoxybenzoate, isobutyl paraoxybenzoate, propionic acid, and sodium propionate.

In one embodiment, it is preferable that the composition comprising a poorly water-soluble or water-insoluble particle is loaded on closer to the tip part of microneedles so as not to lack a sharpness of the tip part thereof. In order to coat a microneedle array with the composition in such condition, for example, a method of immersing a microneedle array in grooves filled with a drug solution (WO 2010/122816) can be used. The amount of the loaded drug and the position of the coating can be properly regulated by controlling the immersion depth (the coated range of a microneedle array). For example, microneedles in a microneedle array are preferably coated from the top to 400 μm therefrom. When the amount of the loaded drug is small, it is enough to coat the microneedles from the tip to 100 μm therefrom.

The microneedle array of the present invention can be manufactured according to known manufacturing methods. For example, the microneedle array of the present invention can be manufactured according to the method described in WO 2012/057345 or WO 2013/162053. In the latter method, microneedles in the manufacture microneedle array have a base section and a shaft section formed on the base section, and form two-stage needles in which the area of the top surface of the base section is larger than the area of the bottom surface of the shaft section. Such shapes allow the composition in larger amounts to be loaded on the upper part of the base section, and also can produce good punctureability.

In one embodiment, the microneedle array of the present invention can be used as a microneedle array formulation.

The second embodiment of the present invention relates to a method of manufacturing a microneedle array having microneedles coated with a composition comprising a poorly water-soluble or water-insoluble particle with a mean particle size of 1 μm or less, 60% by weight or more of water and 2% to 20% by weight of a binding agent.

In one embodiment, the method of manufacturing a microneedle array coated with a composition comprises milling a poorly water-soluble or water-insoluble compound until the mean particle size of the compound is 1 μm or less, preparing a composition comprising the milled particle, 60% by weight or more of water and 2% to 20% by weight of a binding agent, and coating the composition on microneedles.

The poorly water-soluble or water-insoluble compound can be milled according to a well-known method or a method equivalent thereto. For example, it may be milled by dry or wet milling with the milling mechanism described above.

The third embodiment of the present invention relates to a method of manufacturing a microneedle array, which comprises: pouring a composition comprising a poorly water-soluble or water-insoluble particle with a mean particle size of 1 μm or less, water, and a binding agent into a mold with a concave portion forming the form of microneedles, evaporating water to form a microneedle array, and removing the formed microneedle array from the mold. According to the method, a microneedle array having microneedles incorporating a poorly water-soluble or water-insoluble particle with a mean particle size of 1 μm or less and a binding agent can be manufactured.

When microneedles are manufactured using a mold, large particles prevent the composition from spreading throughout the mold because of very small concave portion in the mold, and thus there were problems that a microneedle array having microneedles with smooth surfaces and sharp tips could not be manufactured when performing drying process and that the amount of particles in microneedles varied. On the other hand, a microneedle array having microneedles with smooth surfaces and sharp tips and with no variation in the amount of particle loaded can be manufactured by using a particle with a mean particle size of 1 μm or less.

In one embodiments, the composition comprises 60% by weight or more of water and 2% to 20% by weight of a binding agent. The amount of the binding agent is preferably 3% to 10% by weight. The binding agent may be the same as that used in the first embodiments of the present invention. For example, the binding agent is preferably hyaluronic acid.

The fourth embodiment of the present invention relates to a microneedle array having microneedles incorporating a poorly water-soluble or water-insoluble particle with a mean particle size of 1 μm or less and a binding agent. The microneedles may further comprise various additives. For example, the microneedle array can be manufactured according to the method in the third embodiment of the present invention.

The terms in the third and fourth embodiments in common with the first and second embodiments of the present invention such as microneedle array, poorly water-soluble or water-insoluble particle, binding agent, additive are as defined in the first and second embodiments.

The poorly water-soluble or water-insoluble particle may be obtained by pre-milling a poorly water-soluble or water-insoluble compound until the mean particle size of the compound is 1 μm or less in a similar method to that of the first embodiment.

Pouring a composition into a mold, evaporating water by heating, and removing the formed microneedles from the mold can be performed by a well-known method or a method equivalent thereto.

### Examples

Hereinafter, the present invention is illustrated in Examples in order to make it easy to understand the present invention. The present invention, however, is not intended to be limited thereto by any means.

### 1. Preparation of Drug-Milled Solution and Measurement of Particle Size of Drug

Indomethacin was used as a poorly water-soluble drug. The bulk powder of indomethacin was measured at about 20 points within the same field of view of the microscope. As a result, the mean particle size thereof was 15 μm and SD value was 16 μm. Also, it was showed that the variation in particle size thereof was large. The smallest particle thereof was 2 μm in diameter and the largest particle thereof was 68 μm in diameter.

The bulk powder was milled by planetary mill (THINKY CORPORATION, NP-100). The initial charged composition per batch was indomethacin (1.0 g), 1.25% hydroxypropyl cellulose (HPC-H) (4.0 g), and Zirconia beads (0.1 φmm) (5.0 g), the ingredients were weighted and added into a zirconia container, and they were milled three times at 1700 rpm and −20° C. for 2 minutes. The contents were then transferred to a media separation container and the container was centrifuged once at 2000 rpm and −20° C. for 1 minute to remove zirconia beads and obtain a drug-milled solution.

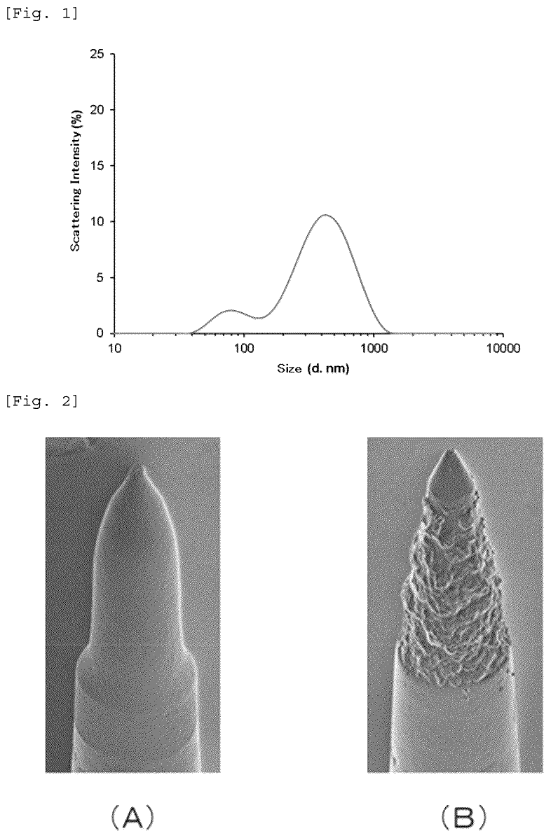

After milling, the drug-milled solution was diluted at appropriate and the mean particle size of the drug was measured by Zetasizer Nano ZS ZEN 3600 (Malvern). The particle size distribution is shown in FIG. 1. The mean particle size thereof was 448 nm.

2. Preparation of Drug Composition and Coating of Microneedles with Drug Composition

To the drug-milled solution prepared in the above 1. were added HPC-H, sucrose and water (MilliQ) in each weight ratio below to prepare a drug composition for coating microneedles (Example). Also, a composition of the same composition as Example but comprising an unmilled product of indomethacin instead of milled indomethacin was prepared as Comparative Example.

TABLE 1

| Ingredient | % by weight |
|---|---|
| Indomethacin | 15.0 |
| HPC-H | 2.2 |
| Sucrose | 5.0 |
| MilliQ | 77.8 |
| Total | 100.0 |

According to the method of immersing a microneedle array in grooves filled with the prepared composition as described in WO 2010/122816, the drug composition was coated on microneedles.

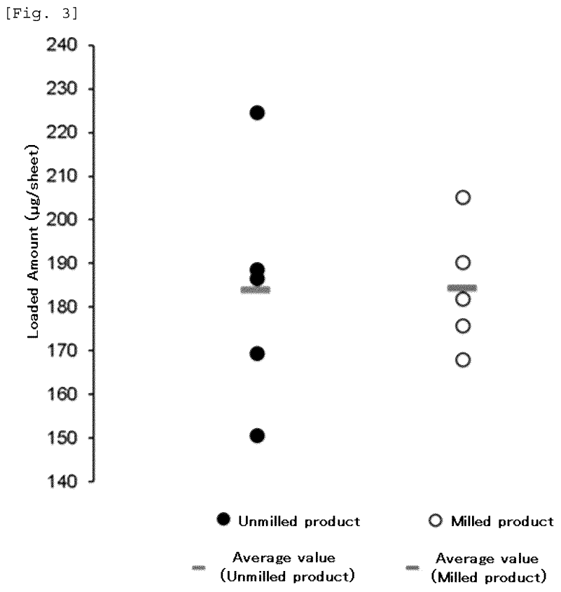

The magnified photograph of microneedles after coating (×600 folds) is shown in FIG. 2. FIG. 2A shows a microneedle coated with the composition comprising milled indomethacin (Example's composition), and FIG. 2B shows a microneedle coated with the composition comprising unmilled indomethacin (Comparative Example's composition).

It was expected that the microneedle coated with the composition comprising milled indomethacin had a smooth surface and sharp tip for good puncturability. On the other hand, it was expected that the microneedle coated with the composition comprising unmilled indomethacin had a rough surface and poor puncturability, and thus the drug composition was removed from the microneedles when punctured.

Then, the amount of indomethacin loaded on the microneedle array was measured by HPLC according to a conventional method.

The measured results of 5 Examples and 5 Comparative Examples are shown in Table 2, Table 3 and FIG. 3. As shown in the results, the microneedle array coated with a composition comprising milled indomethacin showed good reproducibility in the amount of drug loaded. On the other hand, the microneedle array coated with a composition comprising unmilled product of indomethacin is considered to be unsuitable as pharmaceutical formulations due to large variations in the amount of drug loaded.

TABLE 2

| | Loaded amount | Mean | | |
|---|---|---|---|---|
| | (μg/sheet) | (μg/sheet) | S.D. | CV % |
| Example 1 | 182 | 184 | 14 | 8 |
| Example 2 | 168 | | | |
| Example 3 | 190 | | | |
| Example 4 | 176 | | | |
| Example 5 | 205 | | | |

TABLE 3

| | Loaded amount | Mean | | |
|---|---|---|---|---|
| | (μg/sheet) | (μg/sheet) | S.D. | CV % |
| Comparative | 225 | 184 | 27 | 15 |
| Example 1 | | | | |
| Comparative | 169 | | | |
| Example 2 | | | | |
| Comparative | 187 | | | |
| Example 3 | | | | |
| Comparative | 189 | | | |
| Example 4 | | | | |
| Comparative | 151 | | | |
| Example 5 | | | | |

### INDUSTRIAL APPLICABILITY

The present invention can provide a microneedle array which has less variation in the amount of particle loaded and has microneedles with smooth surfaces and sharp tip parts even when coated the microneedles with a composition comprising a poorly water-soluble or water-insoluble particle. Thus, the microneedle array is suitable as pharmaceutical formulations and produces good puncturability.

1. A microneedle array having microneedles coated with a composition comprising:
a poorly water-soluble or water-insoluble particle with a mean particle size of 1 μm or less,
2% to 20% by weight of a binding agent, and
60% by weight or more of water.

2. The microneedle array according to claim 1, wherein the binding agent is one or more selected from the group consisting of a saccharide or a derivative thereof, a protein additive, a polyvinyl alcohol compound, a polyacrylic acid compound, a polyglycolic acid compound, a polyamide compound, a polyester compound, and polyvinyl pyrrolidone.

3. The microneedle array according to claim 2, the saccharide or a derivative thereof comprises one or more saccharides selected from the group consisting of hydroxypropyl cellulose, sodium carboxymethyl cellulose, trehalose and sucrose.

4. The microneedle array according to claim 1, wherein the poorly water-soluble or water-insoluble particle has a mean particle size of 700 nm or less.

5. The microneedle array according to claim 1, wherein the poorly water-soluble or water-insoluble particle has a mean particle size of 500 nm or less.

6. The microneedle array according to claim 1, wherein the poorly water-soluble or water-insoluble particle has a mean particle size of 50 nm or more.

7. The microneedle array according to claim 1, wherein the poorly water-soluble or water-insoluble particle is a drug selected from indomethacin, diclofenac, flurbiprofen, etodolac, fentanyl, lidocaine, apomorphine, donepezil, buprenorphine, naproxen, meloxicam, estradiol, progesterone, methaxalone, cyclosporine, celecoxib, cilostazol, ciprofloxacin, or a salt thereof.

8. The microneedle array according to claim 1, wherein the poorly water-soluble or water-insoluble particle is pre-milled particle.

9. The microneedle array according to claim 1, wherein the amount of the poorly water-soluble or water-insoluble particle is 60% or more relative to the total amount of the composition excluding water.

10. A method of manufacturing a microneedle array, which comprises:
milling a poorly water-soluble or water-insoluble compound until the mean particle size of the compound is 1 μm or less,
preparing a composition comprising the milled poorly water-soluble or water-insoluble compound, 60% by weight or more of water and 2% to 20% by weight of a binding agent, and
coating the composition on microneedles.

11. The method according to claim 10, wherein the milling of the poorly water-soluble or water-insoluble compound is wet milling with at least one mill selected from the group of a jet mill, a bead mill and a planetary mill.

12. A method of manufacturing a microneedle array, which comprises:
pouring a composition comprising a poorly water-soluble or water-insoluble particle with a mean particle size of 1 μm or less, water, and a binding agent into a mold with a concave portion forming the form of microneedles,
evaporating water to form a microneedle array, and
removing the formed microneedle array from the mold.

13. The method according to claim 12, wherein the composition comprises water in an amount of 60% by weight or more and the binding agent in an amount of 2% to 20% by weight.

14. The method according to claim 12, which comprises milling a poorly water-soluble or water-insoluble compound to obtain the poorly water-soluble or water-insoluble particle with a mean particle size of 1 μm or less before pouring the composition into the mold.

15. The method according to claim 14, wherein the milling of the poorly water-soluble or water-insoluble compound is wet milling with at least one mill selected from the group of a jet mill, a bead mill and a planetary mill.

16. A microneedle array manufactured by the method according to claim 12.

17. A microneedle array having microneedles incorporating a poorly water-soluble or water-insoluble particle with a mean particle size of 1 μm or less and a binding agent.
